(19) United States
(12) Patent Application Publication
(10) Pub. No.: US 2011/0030114 A1
(43) Pub. Date: Feb. 10, 2011

(54) ARC FLASH PROTECTION SYSTEM

(75) Inventors: John Merikoski, Wilmette, IL (US); Scott B. Sherman, Northfield, IL (US)

Correspondence Address: MICHAEL J. CHERSKOV, 123 WEST MADISON STREET, SUITE 400, CHICAGO, IL, 60602, US

(73) Assignee: CHICAGO PROTECTIVE APPAREL, INC., Skokie, IL (US)

(21) Appl. No.: 12/850,392

(22) Filed: Aug. 4, 2010

Related U.S. Application Data
(60) Provisional application No. 61/231,215, filed on Aug. 4, 2009.

(51) Int. Cl. A61F 9/06

(52) U.S. Cl. 2/8.7

(57) ABSTRACT

The invention provides up to Category 4 protection against arc radiation, the invented system comprising a protective hood defining a view-panel which confers Category 2 arc flash protection and a region of the hood adapted to encapsulate a Category 2 arc flash protection hard hat or helmet such that the act of encapsulation confers the Category 4 level of protection.

This utility application claims the benefit of U.S. Provisional Application Ser. No. 61/231,215 filed on Aug. 4, 2009, currently pending, the entirety of which is herein incorporated by reference.

### BACKGROUND OF THE INVENTION

1. Field of the Invention

This invention relates to flash protection headgear, and more particularly, this invention relates to a combination hard hat with face shield/protective hood combination that synergistically provides up to Category 4 level of protection.

2. Background of the Invention.

Approximately one person dies each day in the U.S. from arc blasts, and 6-7 are admitted to hospitals for associated injuries.

Arc flashes start with an arcing fault. An arcing fault occurs when current flows through an unintentional path. The current creates an electric arc plasma and releases dangerous amounts of energy. An electric arc is the passage of substantial electrical current through ionized air and gases.

Based on known electrical parameters and work practices, arc exposure intensity can be estimated for the hazard. Arc flash injuries are particularly prevalent among electricians. A myriad of solutions have been proposed to minimize such injuries. One of the common ways to prevent eye and face injuries from arcing is interposing a lens or semi opaque window between the wearer and the arcing source. Semi opaque is defined here as impervious to certain wavelengths or energies that are predetermined as harmful to living tissues.

Arc ratings are the maximum incident energy resistance demonstrated by a material prior to break open or at the onset of a second degree burn. A first degree burn or less is the goal during an electrical arc flash. Exposure energy is expressed in cal/cm². The following are arc hazard exposure levels:

Category 0—N/A;

Category 1—4 cal/cm²;

Category 2—8 cal/cm²;

Category 3—25 cal/cm²;

Category 4—40 cal/cm²;

In perspective, a cigarette lighter placed on the tip of the finger for one second equals roughly a 1 calorie burn.

In light of the foregoing, not all arc energies are alike. Therefore, different arc protection is warranted for different energies. Unfortunately, most protective headgear confers the highest protection only, leading to bulky and hot hoods. Instead of wearing the bulky hoods, workers often do without.

U.S. Pat. No. 3,458,864 to Austin provides a hood with a depending skirt to confer protection against severe heat and fire. The skirt is configured so as to lay tightly against the user's body. While this prevents heat and smoke from entering the hood, this design also does not allow for air exchange, and is therefore relatively hot, particularly in situations where ambient heat and smoke are not an issue.

U.S. Pat. No. 3,427,660 to Raschke provides a system whereby a hat is adapted to receive a curtain along its periphery. This system requires a specially made hat to receive the curtain. However, portions of the hat still remain exposed to the environment.

A need exists in the art for a system to confer protection against arc flashes within a broad range of energies. The system should be light weight for situations where energies do not exceed 600 volts. Furthermore, the system should be synergistic in design such that modules of the system can be combined to confer protection starting at the aforementioned Class 0-1 levels (i.e. up to 600 volts), and all the way up to Class 3-4 levels (5000 volts and beyond). Also, the system should utilize common head gear protection so as to minimize costs.

### SUMMARY OF INVENTION

An object of the invention is to provide a system for arc flash protection that overcomes many of the drawbacks of the prior art.

Another object of the invention is to provide a modularized arc flash protection system. A feature of the invention is a light weight hood adapted to removably receive and completely encapsulate a standard hard hat which itself has a face shield. This encapsulation provides a means for physically isolating the hard hat from the environment situated outside of the hood. An advantage of the invention is that hard hat wearers need not relinquish protection conferred by their own hard hat and face shield when donning the hood.

Still another object of the present invention is to provide a system for conferring arc flash protection over a range of energies. A feature of the invention is a synergistic relationship between a standard hardhat with face shield and a hood, adapted to receive the hard hat with face shield. An advantage of the invention is the wearers need only don the hard hat and shield for Category 1-2 protection but can quickly increase that protection to Category 3-4 with the addition of the hood.

Yet another object of the present invention is providing an arc flash protection device which allows for varying the protection conferred by the device. A feature of the invention is the combination of two lenses to confer Category 4 protection. An advantage of the invention is that Category 1, or 2, or 3, or 4 protection is conferred exclusively of the other categories. Another advantage is that at least two lenses are positioned between the user's eyes and the radiation source at all times that the hat-hood combination is utilized.

Briefly, the invention provides a Category 4 protective hood comprising: a view-panel which confers level 2 arc flash protection; and a region adapted to encapsulate a level 2 arc flash protection hard hat such that the encapsulation forms the category 4 level of protection. In one embodiment of the invention, the hard hat attaches to or otherwise communicates with the hood so as to move in registration with the hood; while in another embodiment, the hard hat moves independent of the hood.

Also provided is a method for varying arc flash protection to a wearer, the method comprising providing a first level of protection capable of conferring up to Category 2 arc flash protection; and providing a second level of protection capable of conferring up to Category 2 arc flash protection, whereby the second level of protection physically encapsulates the first level.

### BRIEF DESCRIPTION OF DRAWING

The invention together with the above and other objects and advantages will be best understood from the following detailed description of the preferred embodiment of the invention shown in the accompanying drawings, wherein:

FIG. 1 depicts a user wearing the invented system, in accordance with features of the present invention;

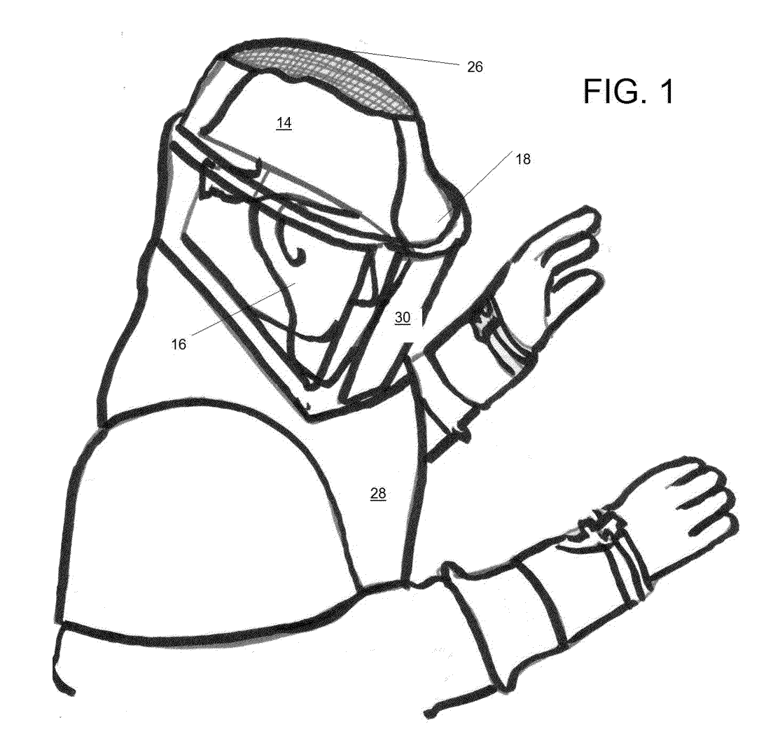

FIG. 2 depicts an elevation view of a modified hard hat, in accordance with features of the present invention; and

FIG. 3 depicts a front view of a hood, in accordance with features of the present invention.

### DETAILED DESCRIPTION OF THE INVENTION

The foregoing summary, as well as the following detailed description of certain embodiments of the present invention, will be better understood when read in conjunction with the appended drawings.

As used herein, an element or step recited in the singular and preceded with the word “a” or “an” should be understood as not excluding plural said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

The invented system for protecting the eyes and face against arc flashes comprises a hard hat adapted to receive a hood. The invented system comprises a light weight paradigm compared to arc protection devices conferring similar protection.

Included below are testing results showing the resiliency of the invented system to various exposure energies. Samples of the arc resistant hoods were tested according to the ASTM F2179-2009 Standard Test Method for Determining the Arc Rating of Face Protective Products. This method evaluates face protective products for ignition, melting and skin burn prediction.

### Hard Hat Detail

A salient feature of the invention is that the hard hat comprises a view shield to confer Category 1-2 arc flash protection. Another salient feature of the invention is that it is adapted to be used with any hard hat or other helmets without the need for special adaptation of the hard hats or helmets. Hard hats with any exterior dimensions are suitable, as the interior of the hard hats are adjustable for head sizes. Usually, head sizes from 6 to 9 are accommodated by hard hats, fireman helmets, police riot helmets, and the like. For illustrative purposes only, the standard dimensions of most hard hats are between 10-13 inches long by 7-9 inches wide by 6 to 8 inches high. However, fireman helmets are also accommodated by the hood, and the standard dimensions of those helmets range from 12-15 inches long, by 9-11 inches wide by 6-13 inches high. These helmets often come with ear and neck protectors so as to confer additional bulk along the dorsal area of the neck, which the hood also accommodates.

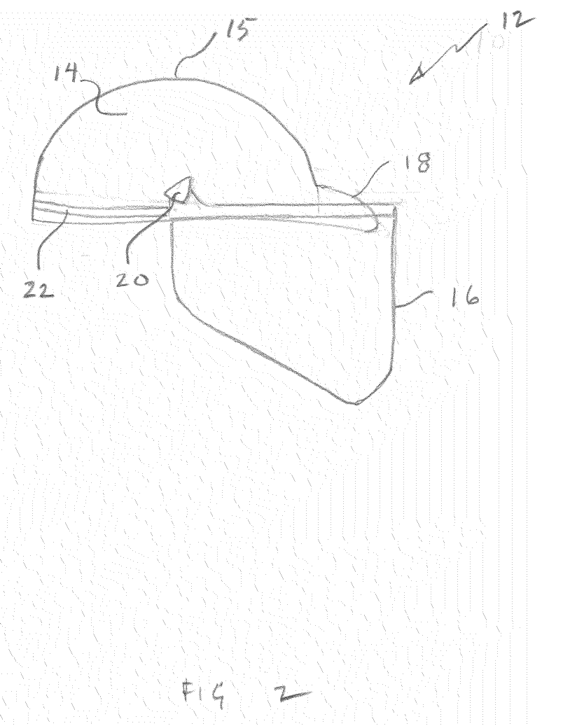

FIG. 2 depicts a hard hat module, designated as element 12, for use in conjunction with the invented protection system. The module 12 comprises a hard hat 14 in hingeable communication with a view shield 16. Generally, the shield cooperates with the front (i.e. visor) 18 of the hard hat 14 so as to allow the user to flip the shield upwardly and over the top of the hard hat 14 when arcing is not present. A proximal point 20 of the shield is in pivotal communication with regions of the hat defining lateral, exterior surfaces of the hat.

Exemplary face shields adapted to receive hard hats are commercially available, such as the Electric Arc Flash Face Shield by Paulson Manufacturing Corporation, Temecula, Calif.

To confer added stability of the shield to the hat, an anchoring substrate, 22, having a first end and a second end, peripherally extends along the back half of the hat with the two ends of the substrate attached to and terminating with the proximal point 20 of the shield. Suitable substrates include reversible deformable substrates such as rubber tubing and elastic bands. Substrates featuring hook-and-pile closure mechanisms (e.g. Velcro® brand closures), snap closures, or simple tie configurations are also suitable.

### Hood Detail

The hood comprises a view panel, which when combined with the aforementioned view shield of the hard hat, confers Category 3-4 arc flash protection. In an embodiment of the invention, the view panel is flexible so as to allow compact stowage of the hood when not in use. In an embodiment of the invention, the view panel comprises a polymer plastic with a thickness of between approximately 1 and 3 mm. However, and as noted throughout this specification, any polymer and thickness which confers category 2 protection is suitable.

Other regions of the hood define a substrate which is opaque to light, and resistant to heat and electricity. As a result of this synergy between the hard hat and the hood, the hood can be constructed of a lighter weight material. Preferably, any portion of the hood withstands up to at least about 40 calories of energy before it fails. This energy amount is akin to Category 4 protection. The hood comprises a flexible view panel, which when combined with the aforementioned view shield of the hard hat, confers Category 3-4 arc flash protection. Other regions of the hood define a substrate which is opaque to light, and resistant to heat and electricity. As a result of this synergy between the hard hat and the hood, the hood can be constructed of a lighter weight material.

The view panel of the hood enables a panoramic view to the wearer. In one embodiment of the invention, the view panel comprises a plurality of planes which are not coplanar with each other so as to confer a field of vision of approximately 180 degrees. In another embodiment of the invention, the view panel comprises a single substrate which is curved or otherwise configured to confer at least about 180 degrees field of vision.

The view panel is made of a transparent material which protects from UV radiation, impacts and scratches. As such, the “transparent” view panel is opaque to the harmful radiation associated with arc flashes. Exemplary material includes polycarbonate plastic (such as Lexan), propionate-containing compounds, or combinations thereof. One embodiment of the view panel comprises Priopionate 0.06, with XLT Enhanced Green color, and about 2 mm thickness. One such view panel is available from Paulson Manufacturing, as discussed above. A periphery of the view panel is attached to the fabric comprising the hood, via heat welding, stitching, or other means. In an embodiment of the invention, only a top peripheral portion of the view panel is attached to the fabric so as to rotate around the axis defined by that peripheral edge. In this case, the wearer can rotate the view shield out of the way when only Category 1 or 2 hazards exist.

In one embodiment of the invention, the weight of the fabric of the hood is about 9-10 oz/yd². It has an approximate ATPV arc rating of 12.4 cal/cm².

The overall arc rating of the hood was calculated as approximately ATPV=19.3 cal/cm². Heat attenuation factor (HAF) was calculated as approximately 90 percent.

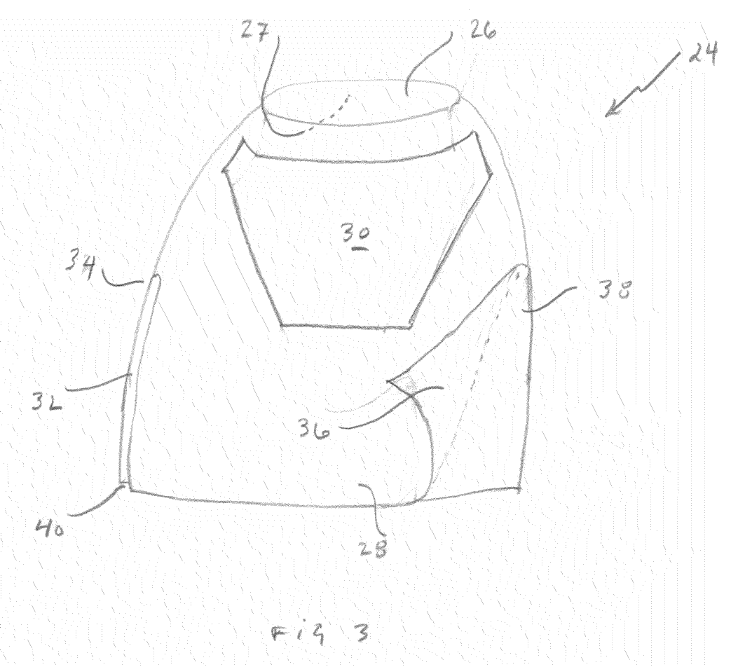

FIG. 3 depicts the hood 24 which is adapted to receive the modified hard hat described supra. In one embodiment, the hood is configured such that no specific interaction, interlocking mechanism, or other means of engagement, aside from frictional interaction, exists between the modified hard hat and the hood. This enables rapid deployment and removal of the hood. Alternatively, hook and pile fasteners, snap fit, or enhanced frictional engagement between the hard hat and the interior of the hood will increase interaction, and therefore coordinated interaction between the hard hat and the hood such that the view panel 30 of the hood lies in registration with the view shield 16 of the hard hat.

The hood 24 comprises a superior region 26 and a depending region 28 defining a skirt. Intermediate these two regions is the view panel 30. FIG. 3 depicts a one piece view panel, which is curved to confer at least 180 degrees of view.

Lateral regions 32 of the hood define longitudinally extending vents or slits to confer unrestricted motion when inserting or removing the modified hard hat. As such, a superior 34 or top portion of the vent defines a contiguous region from a front panel 36 to a rear panel 38 of the skirt. A bottom or inferior region 40 of the vent is not contiguous with the front and back panels so as to be open, as depicted in the folded over region in FIG. 3. These vents provide space to allow the user to insert her hand underneath the hood to adjust the hard hat while it is still encapsulated by the hood, such adjustment including flipping up the hard hat view shield 16 so that the user is only looking through the view panel 30 of the hood.

In operation, a user of the protection system first dons a hard hat, so modified as described supra. With the face shield down, as depicted in FIG. 2, this action confers up to Category 2 protection, per the NFPA 70E-2004 Hazard Risk Category 2 protocol. The Light Transmission Value of the view shield is 70 percent.

If additional protection is warranted, the user continues wearing the hard hat with the view shield in the downwardly (i.e. deployed) position but then slips the hood over the hard hat. The Category 2 protection of the hood is additive to the Category 2 protection of the hard hat so as to confer up to and including Category 4 arc flash protection.

In one embodiment of the invention, when both the hard hat and the hood are worn simultaneously, maximum field of vision is provided inasmuch as the top of the hard hat is in rotatable communication with the inside surface 27 of the apex of the hood. In another embodiment of the invention, the hood moves in registration with the hard hat. If the wearer turns his head to the left, the hood and hard hat move left together. Similarly, a right turn of the head elicits a movement of the hard hat in registration with the hood. No free floating occurs with this embodiment of the invention, and as such, the exterior surfaces of the hard hat communicates with the hood via friction or a hook and pile arrangement (an exemplary embodiment of the later being Velcro® fasteners). Friction between the exterior of the hard hat and the interior surfaces of the hood can be enhanced, or decreased via a scuffing or smoothing respectively, of the exterior of the hard hat.

Either the non-engaging or the engaging embodiments assures that the wearer will have at least two lenses between his eyes and the source of flash or radiation at all times. This two lens configuration also provides added physical protection in situations where projectiles such as tools, slag, bead, etc, hit the outside lens. Also, the rotatable communication embodiment whereby the hard hat is not attached to the hood provides a means for preventing the hood from snagging, bunching-up, or otherwise hindering side to side neck movement of the wearer.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions and types of materials described herein are intended to define the parameters of the invention, they are by no means limiting, but are instead exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the terms “comprising” and “wherein.” Moreover, in the following claims, the terms “first,” “second,” and “third,” are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

The embodiment of the invention in which an exclusive property or privilege is claimed is defined as follows:

1. A Category 4 arc flash protection system comprising:
a) a hood encircling a view-panel which confers Category Level 2 arc flash protection; and
b) a region of the hood adapted to encapsulate a hard hat which itself confers Category Level 2 protection such that the encapsulation forms the Category 4 level of protection.

2. The system as recited in claim 1 wherein the view panel is foldable.

3. The system as recited in claim 1 wherein the hard hat is removably received by the hood.

4. The system as recited in claim 1 further comprising at least two lenses such that when a user is wearing the hood, said at least two lenses are positioned intermediate the eyes of the user and a source of arc flash radiation.

5. The system as recited in claim 1 wherein the hard hat is in rotatable communication with the hood.

6. The system as recited in claim 1 wherein the hard hat moves in registration with the hood.

7. The system as recited in claim 1 wherein the hard hat comprises a substrate which confers category 2 protection for the eyes and face.

8. The system as recited in claim 7 wherein the substrate is a lens comprising a plastic selected from the group consisting of polycarbonate plastic propionate-containing compounds, or combinations thereof.

9. The system as recited in claim 7 wherein the substrate is a plastic lens having a thickness of about 1 to 4 mm.

10. The system as recited in claim 7 wherein the substrate and the view panel are comprised of the same material.

11. The system as recited in claim 1 wherein the view panel is in rotatable communication with the hood.

12. A method for varying arc flash protection to a wearer, the method comprising:
a. providing a first level of protection capable of conferring up to Category 2 arc flash protection; and
b. providing a second level of protection capable of conferring up to Category 2 arc flash protection, whereby the second level of protection physically encapsulates the first level.

13. The method as recited in claim 12 wherein the wearer can remove the flash protection provided by said second level while still wearing said second level of protection.

14. The method as recited in claim 12 wherein the wearer can remove the flash protection provided by said first level while still wearing said first level of protection encapsulated by said second level.

15. The method as recited in claim 12 wherein said first level is moved independently of said second level when worn by the wearer.

16. The method as recited in claim 12 wherein said first level is attached to the second level so as to move in registration with said second level when worn by the wearer.
